(19) United States
(12) Plant Patent Application Publication
(10) Pub. No.: US 2017/0332529 P1
(43) Pub. Date: Nov. 16, 2017

(54) Lemon Tree Named 'CODE 3X97'

(50) ;

(71) Applicant: 2PH Farms Limited, Emerald (AU)

(72) Inventor: Craig Robert Pressler, Emerald (AU)

(73) Assignee: 2PH Farms Limited, Emerald (AU)

(21) Appl. No.: 14/999,477

(22) Filed: May 10, 2016

(51) Int. Cl. A01H 5/08

(52) U.S. Cl. CPC A01H 5/0806
USPC PLT/201

(57) ABSTRACT

‘Code 3X97’ is a new and distinct lemon tree notable for its high quality seedless fruit.

Genus and species: Citrus limon.

Variety denomination: ‘Code 3X97’.

### BACKGROUND AND SUMMARY OF THE VARIETY

The new lemon variety ‘Code 3X97’ originated as an induced mutation of ‘Eureka’ lemon (not patented). Gamma irradiation from a Gammacell 200 (60C) source was applied to bud sticks of ‘Eureka’ in 1998 at Emerald, Queensland, Australia. The irradiated bud sticks were budded onto ‘Carrizo’ rootstock in 1998 at Bundaberg, Queensland, Australia, and the trees that survived were field planted at Bundaberg in 1999. The selection now known as ‘Code 3X97’ was identified as showing consistently fewer seeds than the parent variety, with no significant reduction in fruit size. A comparative trial was planted in 2003, using ‘Code 3X97’ budwood on ‘Benton’ (not patented) rootstock. Successive generations have consistently shown few seeds and have reproduced true to type.

‘Code 3X97’ is a new and distinct lemon tree notable for its fruit, which produces consistently low numbers of seeds. Table 1 shows a comparison of ‘Code 3X97’ lemon to ‘Eureka’ and other similar (unpatented) varieties.

TABLE 1
Comparison of ‘Code 3X97’ to Known Varieties

| Characteristic | ‘Code 3X97’ | ‘3 ELS 0’ | ‘Eureka’ |
|---|---|---|---|
| Seed quantity | Absent to few | Absent to few | Many |
| Mean fruit length (mm) | 76.88 | 74.56 | 84.76 |
| Mean fruit diameter (mm) | 54.52 | 57.16 | 62.84 |
| Fruit length/diameter ratio | 1.41 | 1.31 | 1.35 |
| Rind thickness (mm) | 4.32 | 4.48 | 5.24 |

### BRIEF DESCRIPTION OF THE PHOTOGRAPHS

FIG. 1 shows whole and sectioned fruit of ‘Code 3X97’ lemon;

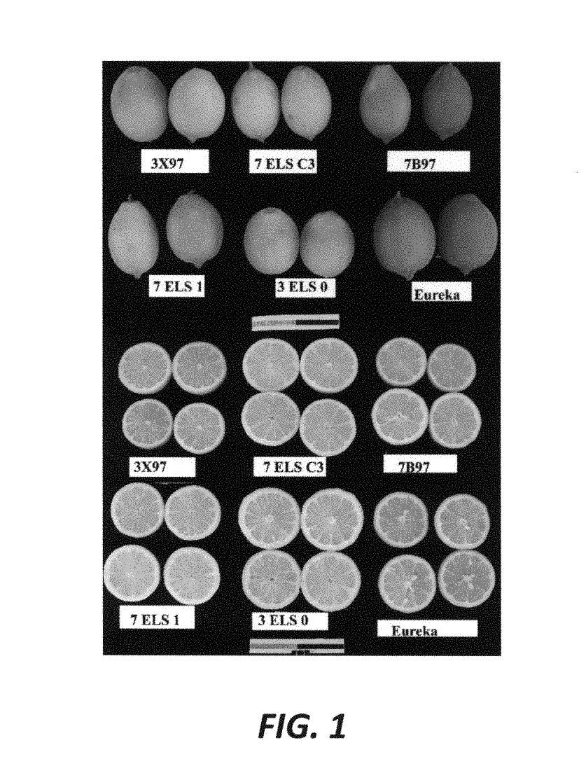

FIG. 2 shows a ‘Code 3X97’ lemon tree; and,

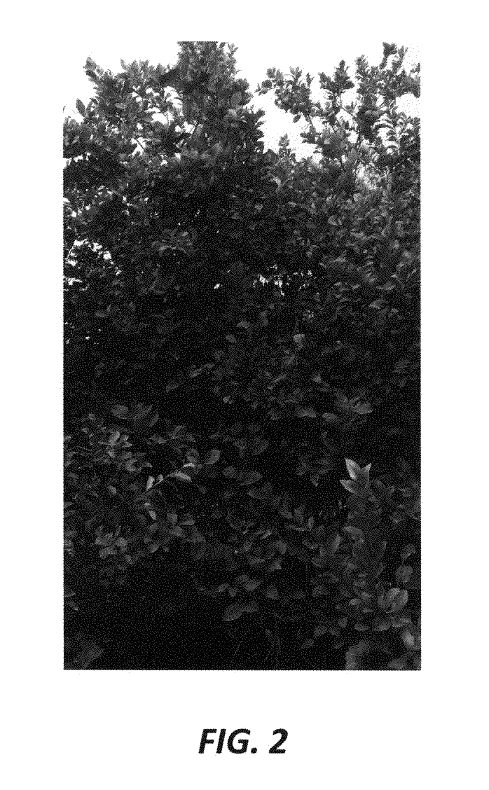

FIG. 3 shows the trunk and branches of a ‘Code 3X97’ lemon tree.

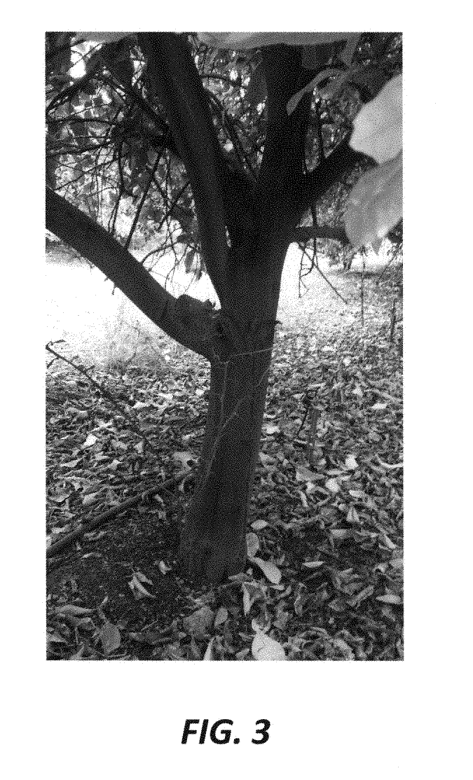

Because lighting conditions can affect the colors shown in photographic illustrations, color characteristics of this new variety should be determined with reference to the observations described herein, rather than from the illustrations alone.

### DETAILED BOTANICAL DESCRIPTION

The following detailed botanical description is based on observations of trees and fruit of ‘Code 3X97’, budded to ‘Benton’ rootstock (not patented) and planted in 2003. Observations were recorded and photographs taken during the 2006 and 2014 growing seasons at Mundubbera, Queensland, Australia. It should be understood that the characteristics described will vary somewhat depending upon cultural practices and climatic conditions, and can vary with location and season. Quantified measurements are expressed as an average of measurements taken from a number of individual plants of the new variety. The measurements of any individual plant, or any group of plants, of the new variety may vary from the stated average. Colors are described with reference to The Royal Horticultural Society Colour Chart.
Tree: Vigor Vigorous Density of canopy Open center Habit Upright spreading Height 4 m Spread 3 m Trunk diameter (at 30 cm above graft) 138 mm Bark texture Smooth Bark color Greyed-orange 163D, greyed-purple N187A Lenticel size 1 mm Lenticel color Greyed-purple N187A Lenticel quantity Many Tendency toward alternate bearing None Winter hardiness Same as ‘Eureka’ Chilling requirement Same as ‘Eureka’ Drought tolerance Same as ‘Eureka’
Branch (fruiting branch located about 1 m above graft union): Length 80 cm Diameter 5.4 cm Crotch angle Approximately 30 degrees Bark color Greyed-orange 163D, greyed-purple N187A Bark texture Smooth Thorns Present on new growth Thorn length Small, 8 to 10 mm Current year shoot Length 87 cm Color Green 140A Blossoms Bud shape Oval Bud length 2.1 mm Bud diameter 1 mm Bud color Yellow-green 144A Quantity of blossoms per cluster 4 to 7 Blossom diameter 21 mm Blossom depth 18 mm Pollen None Sepal length 4.6 mm Sepal width 4.5 mm Sepal shape Arched Sepal margin Smooth Sepal color — upper surface Yellow 2B Sepal color — lower surface Yellow 3B Quantity of petals per flower 4 Petal shape Elongated arch Petal apex 6.1 mm Petal margin Smooth Petal length 16 mm Petal width 5 mm Petal color — upper surface White NN155D Petal color — lower surface White NN155D, violet 84D Date of first bloom Late August Date of full bloom Early September Date of first fruitlet fall November Pedicel length 4.7 mm Pedicel diameter 1.6 mm Pedicel color Yellow-green 151C Pistil quantity per flower 1 Pistil length 11.6 mm Pistil color Orange 26A, yellow 9A, yellow-green 144A Anther quantity per flower 1 Anther length 2.7 mm Anther color Orange 26A Stigma quantity per flower 1 Stigma length 1.8 mm Stigma color Yellow 9A Style quantity per flower 1 Style length 6.4 mm Style color Yellow 1B Ovary quantity per flower 1 Ovary length 4.4 mm Ovary diameter 2.5 mm Ovary color Yellow-green 144A Leaves Length 104.4 mm Width 56.2 mm Length/width ratio 1.85 Blade margin Crenate Leaf shape Oval, weak concave Apex shape Acute Base shape Cuneate Color of upper surface Green 143B Color of lower surface Yellow-green 145A Leaf texture Smooth Petiole length 12.4 mm Petiole diameter 2.9 mm Petiole color Yellow-green N144D Wings None Fruit Quantity per cluster 4 Axial diameter 80.48 mm Apical diameter 59.88 mm Weight 151 g General shape in profile Oval Navel None Rind color Green 140B Oil glands per cm² About 40 Oil gland diameter 1 mm Rind thickness 4.32 mm Ease of peeling Difficult Rind texture Smooth Albedo thickness 3 mm Albedo color Yellow 2C Quantity of fruit segments per fruit 7.7 (average of 25 fruit) Toughness of segment membrane Moderate Juice sac length 7 mm Juice sac shape Cylinder Juice sac length to width ratio 7:1.3 Juice sac color Yellow 2C Juice soluble solids (° Brix) 7.9 Relative harvest maturity (early, late, etc.) Early Harvest window (date range) January through December Seeds present None or very few Stem length 20 mm Stem diameter 4 mm Stem color Yellow-green 144B
Harvest yield: 50 to 100 Tons per hectare.
Market use: Fresh.
Keeping quality: Same as ‘Eureka’.
Shipping quality: Same as ‘Eureka’.

The invention claimed is:

1. A new and distinct variety of lemon tree, substantially as illustrated and described herein
